(19) United States
(12) Patent Application Publication
(10) Pub. No.: US 2022/0386940 A1
(43) Pub. Date: Dec. 8, 2022

(54) ENERGY PRODUCTION AND/OR STORAGE DEVICE COMPRISING A RESERVOIR

(71) Applicants: CENTRE NATIONAL DE LA RECHERCHE SCIENTIFIQUE, Paris (FR); INSTITUT POLYTECHNIQUE DE GRENOBLE, Grenoble (FR); UNIVERSITE GRENOBLE ALPES, SAINT-MARTIN-D'HERES (FR)

(72) Inventors: Michael HOLZINGER, Saint-Laurent du Pont (FR); Jules HAMMOND, Gieres (FR); Andrew GROSS, Grenoble (FR); Jean-Francis BLOCH, Gieres (FR)

(21) Appl. No.: 17/776,842

(22) PCT Filed: Nov. 13, 2020
(86) PCT No.: PCT/EP2020/082150

§ 371 Date: May 13, 2022

(30) Foreign Application Priority Data
Nov. 15, 2019 (FR) 1912792

(51) Int. Cl. A61B 5/00; H01M 8/0297; H01M 8/04082; H01M 8/04225

(52) U.S. Cl. CPC A61B 5/4343; H01M 8/0297; H01M 8/04201; H01M 8/04225; H01M 2250/30; A61B 2560/0285

(57) ABSTRACT

A device for producing and/or storing electrical energy (2), characterized in that the device comprises:
an anode (4),
a cathode (6),
a separator (8) allowing for the transfer of at least one compound capable of triggering and/or enabling a production and/or storage of electrical energy (8), arranged between the anode (4) and the cathode (6), and
at least one breakable, pierceable, and/or deformable reservoir (10) made of a compound capable of triggering and/or enabling a production and/or a storage of electrical energy, said reservoir (10) having means for bringing said compound and said separator (8) into contact with each other; said means for bringing said compound and said separator (8) into contact with each other being, in particular, means for transferring a liquid.

### FIELD OF THE INVENTION

The invention relates to an electrical energy production or storage device allowing for controlled activation by the user, and to the manufacturing and use thereof. In particular, this device may enable the activation and/or control of fuel cells.

### DESCRIPTION OF THE PRIOR ART

Fuel cells function thanks to the transport of ions between two electrodes, generally protons. In order for the cell to function, a medium arranged between the anode and the cathode must be capable of conveying hydroxonium (H₃O⁺) ions, also notated in a simplified version as H⁺, but not electrons. This electrolyte in fuel cells comprises a proton exchange membrane such as in Proton Exchange Membrane Fuel Cells or Polymer Electrolyte Membrane Fuel Cells (PEMFC), and an aqueous liquid allowing for ionic movement. Technological advances have made it possible to develop dihydrogen-dioxygen cells or enzymatic cells, particularly environmentally-friendly glucose-dioxygen enzymatic cells (cf. WO2018/185417 on behalf of the CNRS [French National Scientific Research Center]). Such cells are compact and can work at low temperatures (less than 80° C.), possibly with a polymer electrolyte (PEMFC) or an aqueous electrolyte (aqueous solution, biological fluids). They can be used not only in a stationary mode, but also in a portable mode. These cells can therefore be suitable for generating small currents and for household and/or personal uses. However, the activation and/or control and possibly deactivation of such devices remains problematic. So far, control is achieved by opening the electrical circuit. But any contact of these parts of the cell (anode, cathode, membrane) with a liquid may damage these parts, particularly when they contain biological materials (plant materials, enzymes, etc.). Consequently, the liquid required for operation of the cell is advantageously added to the membrane at the time the cells first function. This is generally done by an external addition of liquid to the membrane of the device, in particular by using a pipette, as described in patent application US2011287328A1 by Sony, or by using a reservoir as described in patent application US2010/0297477 by Power Knowledge Ltd. There are also numerous problems associated with such devices: first, the user must have access to an (aqueous) liquid source of sufficient purity and quality for the required use and proper operation of the cell. The use must also be able to quantify the volume to add. This requires the availability of a separate measurement device such as a disposable pipette or dosette, which increases the costs and environmental impact of the device. In addition, the device must comprise a means for putting the liquid on the membrane. The fact that the membrane may be accessible from the outside notably implies additional contamination problems. Lastly, cells of this type are difficult to store because they may be sensitive to the relative humidity of their environment. There is therefore a need for devices for producing electric current that do not have the disadvantages mentioned above.

### DESCRIPTION OF THE INVENTION

The invention therefore relates to a device for producing and/or storing electrical energy, particularly electrochemical energy, characterized in that the device comprises:
an anode,
a cathode,
a separator, such as a diffusion layer, allowing for the transfer of at least one compound capable of triggering and/or enabling a production and/or storage of electrical energy, arranged between the anode and the cathode, and
at least one breakable, pierceable, and/or deformable reservoir made of a compound capable of triggering and/or enabling a production and/or a storage of electrical energy, said reservoir having means for bringing said compound and said separator into contact with each other; said means for bringing said compound and said separator into contact with each other being, in particular, means for transferring a fluid and, in particular, a liquid.

The reservoir (“storage tank” in English), like any reservoir, defines a location separate from the energy production and/or storage location where the contents of the reservoir are used. It may adjoin or even straddle this location, but it does not define a strictly identical location. In other words, the reservoir and the electrochemical cell are separate and preferably separated parts. This reservoir may advantageously separate the contents of the reservoir from the anode and the cathode, in whole or in part.

The reservoir and its contents therefore participate in the transition from an inactive to an active state of the device. The deformation of the reservoir and the release of its contents create an aptitude for activation of the device. The deformation of the reservoir may therefore make it possible to directly trigger energy production or storage, or create an intermediate activation state of the device. Indeed, the device may additionally comprise one or more activation means, the configuration of which is chosen to either activate or increase or decrease or shut down (or deactivate) the energy production and/or storage. For example, the device may also comprise a switch that may be operated advantageously by the user. Such a switch may comprise, for example, a circuit-breaker such as a removable tab. This switch can then be operated by the user before or after the compound for triggering an electrical energy production or storage and the separator have been brought into contact with each other. Thus, activation may be direct or it may occur in several steps. For example, activation may occur initially by pressure on the reservoir (and possibly the release of fluids into the reservoir) and then by the removal of a circuit-breaker tab by the user. The subject matter of the invention also involves the activation of the device by the user, particularly in a sequential manner in several steps or in a single step.

Advantageously, in the device according to the invention the anode and/or the cathode comprises an enzyme.

The reservoir is breakable, pierceable, and/or deformable in whole or in part. For example, it may comprise a part which can be destroyed by the user. It may comprise a container in the form of, for example, a shell (advantageously deformable and/or flexible) having an opening and retention means that closes the opening of the shell or container. This shell, for example a potentially semi-spherical dome, therefore forms a cavity containing the compound, and comprises an opening. The retention means is advantageously a separating layer or film blocking the opening of the shell or container and forming together therewith a closed and preferably impermeable receptacle. This type of reservoir is also known by its English name “blister pack” or simply “blister.” It may be characterized in that the opening surface area is proportionally large, for example 25 to 45%, with respect to the total surface area of the reservoir. However, the invention is not limited by such a feature, which may be larger or smaller. The retention means retain the compound inside the reservoir and particularly separate it, at least in part, from the anode and the cathode.

The choice of material or materials used to make the shell and the separation layer depends on a number of factors. For example, a layered material comprising a layer of resistant material is put in place if the shell is intended to contain a liquid, a semi-liquid, a gel, or substances that can move around. It is also possible to choose materials that are inert [3] with respect to the material stored in the reservoir or materials absorbing water vapor, oxygen (or both) to control the atmosphere inside the reservoir and thus preserve the contained product (increased stability over time) [4]. One or more materials having the lowest environmental impact may also be chosen [2]. The material is generally a thermoformed material [1] and may be chosen from the group of materials consisting of:
polyvinyl chloride (PVC),
polychlorotrifluoroethylene (PCTFE) (and particularly ACLAR™)
cyclic olefin copolymer (COC),
polyethylene (PE),
ortho-phthalaldehyde (OPA),
aluminum,
heat seal lacquers (HSLs).
vinyl acetate copolymers of maleic acid and vinyl chloride (VMCH),
polyvinylidene chloride (PVDC),
polyethylene terephthalate (PET),
glycol-modified polyethylene terephthalate (PETG),
cyclic olefin copolymers (COCs),
polypropylenes; and
mixtures thereof.

The reservoir may also comprise laminates and in particular laminated sheets of these materials. These components have advantageous properties of chemical and/or moisture resistance. For example, a PCTFE, a PVC covered with PVDC or a polypropylene is particularly well-suited for the reservoir, and in particular the shell, owing to its ability to act as a barrier to air and moisture. In addition, materials combining a laminated sheet of aluminum and HSL or aluminum and VMCH are also preferred, in particular for making the retention and/or separation means. Alternatively, a material based on cellulosic fibers (such as paper or cardboard) is an advantageous alternative to these materials from an environmental standpoint and may be considered. In particular, this material may be combined with films such as plastic films and particularly a film in one of the thermoformed materials mentioned above.

The size of the reservoir may be determined by the quantity of the compound (liquid, for example) to be released. The term liquid comprises not only a compound or a composition, but also encompasses semi-liquids (for example, a viscous compound or composition) as well as gels.

The compound for triggering an electrical energy production and/or storage contained in the reservoir may be a liquid, a solid, or a gel, preferably an aqueous liquid, that is, one primarily based on water or simply just water (which may have different degrees of purity: distilled water, pure water, tap water, etc.).

The compound may be a composition comprising at least one compound capable of triggering an electrical energy production and/or storage and comprise or consist of an electrolyte (phosphate buffer containing sodium or ammonium sulfate, an enzyme (for example glucose oxidase and/or FAD dehydrogenase for the anode and laccase and/or bilirubin oxidase for the cathode), an electronic exchange mediator (for example ABTS, 1,2 gold 1,4 naphthoquinone, phenanthroline quinone, or pyrene and one of the derivatives thereof), a cofactor (for example an NAD⁺/NADP⁺ or flavin coenzyme such as FAD (Flavin Adenine Dinucleotide), or FMN (Flavin mononucleotide), a promoter (for example protoporphyrin IX) of a substrate (for example glucose) and/or an enzyme orientation molecule, that is, an enzyme or a molecule capable of acting on a parasitic or toxic substance (for example, catalase for breaking down the hydrogen peroxide produced by the enzyme glucose oxidase).

The means for bringing the compound (for example, a liquid) and the separator into contact with each other may comprise the choice of a material allowing for a break, preferably a break made at a particular point or a special area of the reservoir, due to the user applying sufficient pressure to a part of the reservoir. This is the case of blister packs for medicinal tablets in which pressure applied to the shell of the blister pack and the deformation thereof cause the film closing the receptacle—which is not as strong—to break and make the tablet available [cf.5-9]. This arrangement may be suitable for the device according to the invention, since liquids are not compressible.

According to another variant of the invention, the means for bringing the compound and the diffusion layer into contact with each other may comprise piercing means, which may potentially comprise at least one part having a cutting or pointed end. Thus, these piercing means may, for example, be in the form of needles, blades, protuberances, or spikes. These means are placed in front of the surface to be pierced, but may also be placed inside or outside the reservoir. When the reservoir is a blister pack or a shell, the piercing means are advantageously arranged so as to be actuated by the user pressing on the outside surface of the shell.

Said means for bringing the compound and the diffusion layer into contact with each other favorably comprises a duct toward and/or a projection from the separator, said projection or said duct preferably being configured to come into contact with a part of the reservoir, and preferably, when the reservoir is in the form of a blister pack, directly in contact with the retention means. Thus, the means for bringing the compound into contact with the separator may simply be juxtaposition and/or direct contact of at least a part of the separator with the reservoir, and more particularly with a breakage area thereof, if it exists. Thus, the separator is advantageously configured to comprise a part extending beyond the electrodes (i.e., of the anode and the cathode) and positioned in front of the (provided) breakage area of the reservoir.

The separator may be a diffusion or migration layer. It may be a simple space between the anode and the cathode, a space intended to be filled with a compound capable of triggering electrical energy production and/or storage. It may also be integrated into or separate from the surface of one or both of the electrodes. Advantageously, the separator comprises or essentially consists of a material adapted for an electrolyte substrate function. This material comprises woven or nonwoven fibers (cotton, nylon, polyesters, glass), ceramic, and natural substances (rubber, asbestos, wood). It may comprise a polymer material such as polyethylene, polypropylene, poly (tetrafluoroethylene), and/or polyvinyl chloride, such as the ionomer Nafion™, a perfluorinated polymer made by Dupont. It may also be a gel, such as an ionic gel and/or a hydrogel, or an item making it possible to create a gel.

However, for household use or use by the general public, the use of a porous membrane is also envisioned, particularly one made of cellulosic fibers, such as a sheet of paper and in particular porous paper such as blotting or filter paper. The weight of this paper may be chosen advantageously within the range of 10 to 300 g/m⁻², preferably 50 to 150 g/m⁻².

The thickness of the separator is generally low, but it must be adapted to the desired use. Thus, a thickness of 2 mm to 10 μm, particularly 1 mm to 10 μm, preferably 300 to 150 μm (for example 190 μm) can be used. Thus, a paper with a weight of 97 g/m⁻² constituting the separator is sandwiched between the two electrodes. The separator may comprise a single or a plurality of layers/sheets of material.

According to a particular embodiment of the invention, the reservoir of the device may comprise one or more compartments. As will be described below, these compartments may comprise identical contents or contents that differ from each other. However, it is particularly advantageous to use this configuration to make it possible to release individual doses of compounds or to prepare a composition comprising a mixture of components that are unstable over time.

According to another particular embodiment of the invention, the device comprises at least one other reservoir, this other reservoir comprising said compound capable of triggering an electrical energy production and/or storage, or another compound that may or may not be capable of triggering an electrical energy production and/or storage. As will be described (see below), these reservoirs may comprise identical or different contents and be the same or different sizes, depending on the desired purpose. When the contents are identical, said other reservoir may be used as a recharge of the device. These reservoirs may be arranged one after the other, or positioned on either side of the anode and cathode.

According to another particular embodiment of the invention, the device may comprise means for (partial, temporary, and/or final) deactivation of the electrical energy production and/or storage. Such means may advantageously be in the form of another reservoir having a structure similar to that described in the present application. Such means comprise means for sucking the compound capable of triggering an electrical energy production and/or storage and therefore make it possible to suck up and store said compound. The suction means may, for example, be the presence of a partial vacuum in the reservoir associated with means of opening the reservoir, allowing for suction inside the reservoir. These means may also comprise an absorbent substance for sucking up a liquid by capillary action. Other aspects of this particular embodiment are described below.

According to a particular aspect of the invention, the device may have more than one reservoir or one compartment, the contents and configuration of which are chosen to either activate or increase or decrease or shut down (or deactivate) the energy production and/or storage. Such devices are described in detail below. Thus, in one instance, the invention relates to a device as described in the present application comprising means for increasing, decreasing, deactivating, and/or reactivating the electrical energy production and/or storage.

The anode and cathode of the device according to the invention are electrodes adapted for use as fuel cells. Naturally, the device according to the invention may also comprise a series of anodes and cathodes in a stack. The anode and cathode may be made of metal with, for example, a cathode made of silver and an anode made of chrome-plated nickel. However, it is preferable for the anode and the cathode to be of a type suitable for biofuel cells and/or enzymatic biofuel cells. These bioelectrodes (anode or cathode) may comprise a substrate consisting of, or having deposited on the surface thereof, carbon nanotubes, a redox mediator, and an enzyme. These electrodes may be multilayered and advantageously comprise:
a carbon layer having a high active specific surface area, such as a layer of carbon nanotubes, carbon black, or carbon in the form of aggregates or gel, carbon-based materials with high specific surface areas such as carbon nanotubes or mesoporous carbon matrices, in particular those obtained from magnesium oxide MgO (see Inagaki et al. “Templated mesoporous carbon: Synthesis and applications” Carbon 107 (2016) 448-473) are preferred.
a mediating and/or orienting layer; and/or
an enzyme layer for catalyzing the oxidation or reduction reactions of the fuel.

The layers may be arranged in succession on an electrically conductive material which may constitute the substrate of these layers, or be itself deposited on an inert substrate.

The conductive material may be vitreous carbon, pyrolytic graphite, in particular “Highly Ordered Pyrolytic Graphite” (HOPG), gold, platinum, and/or indium and tin oxide. Preferably, the material consists of vitreous carbon or pyrolytic graphite. As presented in the examples, the bioelectrodes may comprise a sheet of carbon nanotubes functionalized by an enzyme and preferably a mediator.

The nanotube sheet is advantageously applied to a conductive material belonging to a microporous gas-diffusion electrode comprising a Gas Diffusion Layer (GDL), a layer which generally comprises carbon fibers. Nanotube sheets adapted to this use are commercially available or may easily be made using a suspension of nanotubes in a solvent such as N, N-dimethylformamide (DMF), sonication (e.g. 30 minutes), and filtration (PTFE filter by the company Millipore PTFE (JHWP, pore size 0.45 μm, ø=46 mm). This method is described in detail in Gross et al. (2017) “A High Power Buckypaper Biofuel Cell: Exploiting 1,10-Phenanthroline-5,6-dione with FAD-Dependent Dehydrogenase for Catalytically-Powerful Glucose Oxidation” ACS Catal. 2017, 7, 4408-4416.

When the fuel of the biofuel cell is glucose, the enzyme capable of catalyzing the oxidation of glucose at the anode is preferably a Glucose DeHydrogenase (GDH) catalyzing the reaction:

D-glucose+acceptor→D-glucono-1,5-lactone+reduced acceptor.

The acceptor, or co-factor, is generally an NAD+/NADP+ or flavin coenzyme, such as FAD (Flavin Adenine Dinucleotide), or FMN (Flavin mononucleotide) which is bound to the GDH. An especially preferred glucose dehydrogenase is Flavin Adenine Dinucleotide-Glucose Dehydrogenase (FAD-GDH) (EC 1.1.5.9). The term FAD-GDH encompasses native proteins and the derivatives, mutants, and/or functional equivalents thereof. In particular, this term encompasses proteins that do not substantially differ in structure and/or enzymatic activity. Thus, a GDH enzymatic protein having a sequence of amino acids having at least 75%, preferably 95%, and even more preferably 99% identity with the GDH sequence or sequences as registered in databanks (for example SWISS PROT) may be used for the anode in combination with a cofactor. An FAD-GDH of Aspergillus sp. is especially preferred and effective, but other FAD-GDHs coming from Glomerella cingulata (GcGDH), or a recombinant form expressed in Pichia pastoris (rGcGDH), could also be used. It is also possible to use an anode by using an oxidoreductase enzyme (EC 1.1.3.4) of the glucose oxidase (GOx, GOD) type, which catalyzes the oxidation of glucose in hydrogen peroxide and in D-glucono-δ-lactone. This enzyme is also bound to a cofactor like FAD (Flavin Adenine Dinucleotide). An especially preferred glucose oxidase is Flavin Adenine Dinucleotide-Glucose oxidase (FAD-GOx). This term encompasses native proteins and the derivatives, mutants, and/or functional equivalents thereof. In particular, the term FAD-GOx encompasses proteins that do not substantially differ in structure and/or enzymatic activity. Thus, a GOx enzymatic protein having a sequence of amino acids having at least 75%, preferably 95%, and even more preferably 99% identity with the GOx sequence or sequences as registered in databanks (for example SWISS PROT) may be used for the electrode according to the invention in combination with a cofactor. An FAD-GOx extracted from Aspergillus niger is especially preferred. FAD-GDH has a greater activity than glucose oxidase and therefore a higher catalytic current. This is very attractive for the purpose of increasing the power generated in enzymatic biofuel cells. It should be noted that, contrary to Glucose Oxidase, the FAD-GDH enzyme does not produce hydrogen peroxide. Due to its oxidizing properties, hydrogen peroxide may entail drawbacks for the stability of biofuel cells (membrane, stability of enzymes in the cathode, etc.).

In the particular case of glucose biofuel cells, the reduction of dioxygen takes place in the cathode. The enzyme or enzymes that can be used may be chosen from the group consisting of the laccase enzyme, advantageously associated with the mediator ABTS (2,2′-azino-bis(3-ethylbenzthiazoline-6-sulphonic acid) or a mediator/“orienter” such as pyrene, naphthalene, anthracene, or anthraquinone, or Bilirubin oxidase associated with the promoter protoporphyrin IX or mediator ABTS (2,2′-azino-bis(3-ethylbenzthiazoline-6-sulphonic acid).

The device according to the invention advantageously comprises an item, or electrical circuit, connected to an electrical consumer that allows an electric current to flow. As described above, this circuit may include a switch.

Although the device according to the invention is particularly well suited for biofuel cells and/or the corresponding electrodes, the device is not in any way limited to this embodiment.

Depending on the preference, the device is portable and preferably self-contained. A self-contained device is a device that is contained within itself and which advantageously does not include removable parts. As such, the device advantageously comprises a housing or “casing” that can protect the anode and cathode while still allowing the user to access the deformable part of the reservoir.

According to a preferred aspect of the invention, the device is a device that is stand-alone, isolated, and/or independent of external inputs other than gas. In particular, it does not require any liquid and/or fuel inputs.

The invention also relates to a method for manufacturing the device according to the invention. This method comprises the steps for connecting the elements of the device as described in a functional way. In particular, the method comprises the arrangement (i) of at least one compound capable of triggering an electrical energy production or storage between the anode and the cathode, and (ii) a reservoir for a liquid capable of triggering an electrical energy production or storage, and means for opening the reservoir; and a means of bringing the liquid with the separator into contact with each other.

The invention also relates to a device incorporating the device according to the invention as a current generator.. As a non-limiting example, let us mention a medical test (for measuring or diagnosing) conducted by an individual on him/herself or on a patient at the treatment location or nearby, that is, at the time and location of care (also known as “Point-Of-Care Testing” or POCT). For example, such a test can be an ovulation and/or pregnancy test. The device may also be a non-medical test and may comprise an electronic device or packaging capable of integrating a container of the liquid in its design in order to activate energy production.

The invention also relates to the use of a device according to the invention to produce electrical energy and advantageously in the manufacturing of an electrical or electronic device as described in the preceding paragraph.

Another aspect of the invention is a kit or “kit-of-parts” for making an energy production device such as an electrochemical cell, for example a battery. Such a kit comprises the device according to the invention accompanied advantageously by usage instructions.

### BRIEF DESCRIPTION OF THE FIGURES

The invention will be better understood through the following description, provided solely as an example and given in reference to the appended drawings, in which:

FIG. 1 is a diagram of the different steps illustrating the operating principle of an electrical energy production device according to a first embodiment,

FIG. 2a is a cross-sectional schematic view of a reservoir comprising means for releasing the solution according to a first variant,

FIG. 2b is a cross-sectional schematic view of a reservoir comprising means for releasing the solution according to a second variant,

FIG. 2c is a cross-sectional schematic view of a reservoir comprising means for releasing the solution according to a third variant,

FIG. 3 is a cross-sectional schematic view of a reservoir with a plurality of compartments according to a first variant,

FIG. 4 is a cross-sectional schematic view of a reservoir with a plurality of compartments according to a second variant,

FIG. 5 is a schematic representation of an electrical energy production device according to a second embodiment of the invention,

FIG. 6 is a schematic representation of an electrical energy production device according to a third embodiment of the invention,

FIG. 7 is a schematic representation of an electrical energy production device according to a fourth embodiment of the invention,

FIG. 8a is a schematic representation of a combination of two reservoirs that can be used in an electrical energy production device according to the invention,

FIG. 8b is a schematic representation of a combination of two reservoirs that can be used in an electrical energy production device according to the invention,

FIG. 8c is a schematic representation of a combination of three reservoirs that can be used in an electrical energy production device according to the invention,

FIG. 9 is a first exploded perspective view, as seen from the front and below, of an electrical energy production device according to the invention of example 1,

FIG. 10 is a second exploded perspective view of FIG. 9, viewed this time from the side and slightly above, of the electrical energy production device of example 1,

FIG. 11 is a curve showing the energy production of the electrical energy production device of example 1.

### DETAILED DESCRIPTION

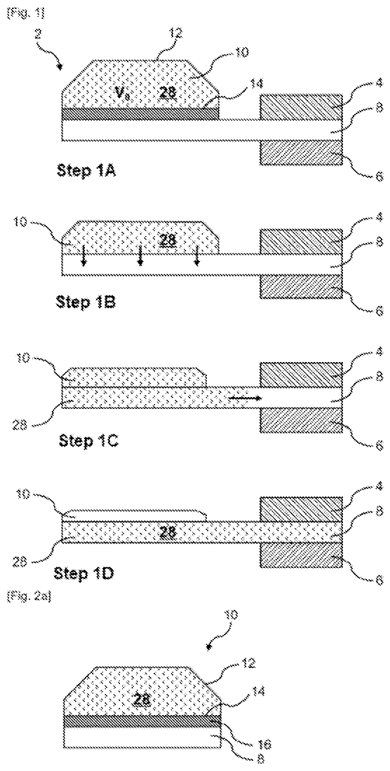

We shall now refer to FIG. 1 schematically showing an electrical energy production device according to the invention. Initially, we shall describe different variants of this device before describing the implementation and energy production method.

Anode and Cathode

The electrical energy production device 2 comprises an anode 4 and a cathode 6. In order to ensure that the oxidation-reduction reaction for producing electrical energy occurs, the anode 4 and the cathode 6 are made of materials enabling an ion exchange. The anode and cathode must have specific properties (thickness, conductivity, surface resistance) chosen as a function of the application. These parts may be impregnated with enzymes and mediators.

For example the anode 4 and the cathode 6 comprise the sheets of nanotubes, and in particular sheets composed of multi-walled carbon nanotubes (MWNTs) as described above. In the case of glucose fuel cells, the sheet of nanotubes is impregnated with mediators and enzymes allowing the glucose to oxidize at the anode and the oxygen in air to be reduced in water at the anode. For example, the anode 4 may comprise the enzyme glucose oxidase and/or FAD dehydrogenase for glucose oxidation, as well as naphthoquinone and/or phenanthroline quinone as redox mediator transferring electrons to the electrode.

As for the cathode, it comprises the enzyme laccase, bilirubin oxidase, and ABTS as mediator.

Separator

A diffusion layer 8, or separator, is placed between the anode 4 and the cathode 6. The latter enables diffusion or transfer of a solution triggering electrical energy production by oxidation-reduction between the anode and cathode.

The transfer can take place by what we shall call a diffusion layer. The diffusion layer 8 can, for example, be a simple space or, more advantageously, it may comprise or consist of a paper-type material in which the solution triggering the oxidation-reduction can be diffused by capillary action. A compromise must be struck between its thickness and its alveolar capacity (void volume).

This diffusion layer 8 forms a separating layer between the anode and the cathode and may also constitute, at least in part, the diffusion substrate for the electrolyte.

Reservoir

The electrical energy production device of FIG. 1 also comprises at least one reservoir 10, which is preferably deformable. In this variant, reservoir refers to a means of retaining at least one liquid (including a semi-liquid or a gel) enabling an ion exchange between the anode and the cathode and the production of electricity by soaking the diffusion layer 8 once it is released. The diffusion layer 8 is positioned between the anode 4 and cathode 6 and may comprise a portion (a tab, for example) extending to the outside. All or part of it is in contact with the fluid, with a retention means 14, which may consist of a separation layer, blocking the reservoir 10. The fluid contact may or may not be direct. The reservoir 10 comprises a shell 12 forming a cavity in which the liquid is enclosed and a separation layer blocking the opening of the shell 12. This device is also known by its English name “blister pack.”

The shell 12 is advantageously deformable. It may be made of a material chosen from among polyvinyl chloride (PVC), a material made by fluorinated-chlorinated resins, cyclo-olefin polymers (COPs), cyclo-olefin copolymers (COCs), polyethylene (PE), oriented polyamides (OPAs), aluminum (Al), aluminum in combination with heat seal lacquer (HSL), or aluminum in combination with a vinyl chloride maleic acid vinyl acetate copolymer (VMCH).

The choice of material or materials used to make the shell 12 may depend on a number of factors. For example, if the shell is intended to contain a liquid or substances that can move around, a barrier layer is put in place. It is also possible to choose materials that are inert with respect to the material stored in the reservoir 10 or materials absorbing water vapor, oxygen (or both) to control the atmosphere inside the ampoule 10 and thus preserve the contained product (increased stability over time). One or more materials having the lowest environmental impact may also be chosen.

The liquid held inside the reservoir 10 may, for example, be an aqueous glucose solution that interacts with the enzymes mentioned above to allow for the exchange of protons between the cathode 6 and the anode 4. The advantage of this device is that no external input other than oxygen is required for the electrical energy production device 2 to function.

The liquid remains enclosed in the reservoir 10 until the production of an electric current is desired. Isolation of the liquid prevents contamination between the electrical energy production device 2 and the environment of this device.

The retention means 14 blocking the opening of the shell 12 keeps the liquid in the ampoule. It may be made of one or more of the materials mentioned above. For example, it may consist of a composite film composed of an aluminum layer (possibly coated with a protective layer of polyethylene terephthalate (PET)) and a sealing layer (made of polypropylene or polyethylene, for instance). A solution based on biodegradable materials, such as paper or a film made of biodegradable PVC such as ECOmply™ sold by Bilcare Research AG, Hochbergerstrasse 60B 4057 Basel, Switzerland, is preferred.

The retention means 14 is capable of breaking under pressure in order to release the contents of the reservoir 10. FIG. 1 describes the operating principle of an electrical energy production device 2.

In step 1A, the device is in the inactive state. A liquid (or a semi-liquid or a gel) 28 is confined to the reservoir 10. In step 1B, the liquid 28 is released from the reservoir 10 by breakage of the retention means 14. The liquid 28 is then released. In step 1C, the liquid 28 spreads into the diffusion layer 8. In step 1D, the liquid 28 reaches the portion of the diffusion layer located between the anode 4 and the cathode 6. The presence of this liquid then allows for the ion exchange between the anode 4 and the cathode 6, which induces the production of electricity by an oxidation-reduction reaction, the type of which may vary depending on the chosen electrochemical cell.

Devices for Breaking the Separation Layer

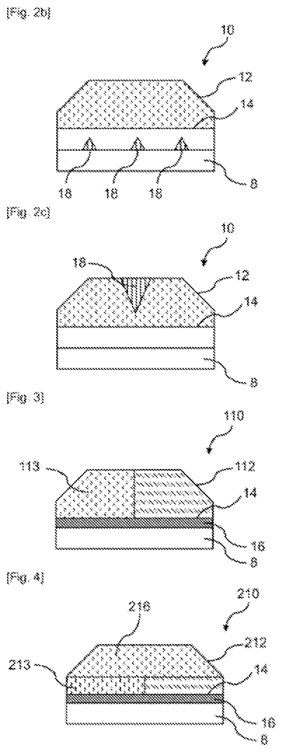

The retention means 14 is capable of breaking under pressure in order to release the contents of the reservoir 10. Several options may be implemented in order to do this:
As shown in FIG. 2a, it is possible, for example, to cause a seal 16 providing a seal between the shell 12 and the diffusion layer 8 to break when a pressure is exerted on the shell 12. In this case, the increased pressure inside the ampoule breaks the seal 16. The seal is sized and the material thereof is chosen so that the seal breaks as soon as the pressure reaches a threshold value. It is also possible to provide weak points in the seal 16 to facilitate this breakage;
It is additionally or alternatively possible to provide piercing means 18 in the retention means 14. These piercing means may be in the form of needles or spikes which may be arranged inside or outside the reservoir 10. FIG. 2c shows a piercing means 18 placed inside the chamber of the ampoule 10 (the number may also vary). FIG. 2b shows the case in which three piercing means 18 (their number may vary) are placed in front of or in the seal outside the chamber of the ampoule 10.

If piercing means 18 are used, it is preferable to provide a gap between these means and the retention means 14 to avoid accidental piercing. In this way, piercing will only occur when a sufficiently strong pressure is exerted.

An external pressure exerted on the shell 12 deforms the shell in order to release the contents of the reservoir 10. This pressure may be exerted by a user or by automated hydraulic or pneumatic pressure means.

Multi-Compartment Reservoir

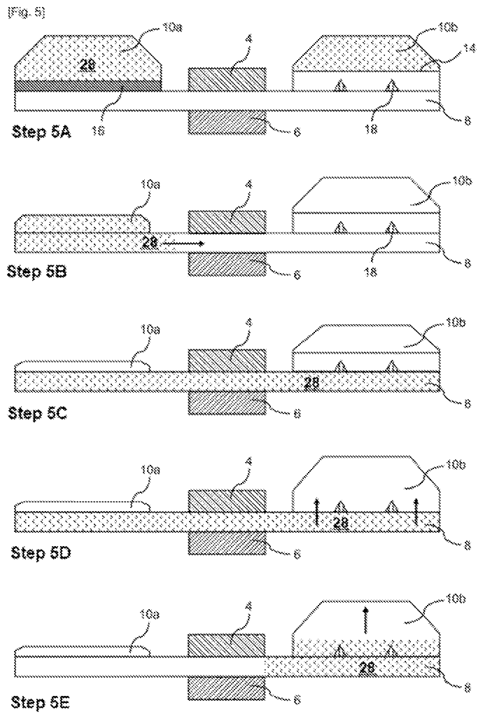

The reservoir 10 of the device according to the invention may comprise one or more compartments. Indeed, it may be advantageous to separate the components for triggering the production of electricity. For example, it is possible to separate different chemical compounds required for operation of the cell. However, combination in the same reservoir for a long period of time could cause undesirable reactions, such as deterioration. It is also possible to preserve biomolecules in a particular state (dry, wet, as a gel, powder, etc.) in one or more compartments, with breakage of the various compartments making it possible to obtain the energy production compound (for example, an item preserved dry and then solubilized by a solvent present in another compartment). The use of a plurality of compartments not only preserves the various components, but also provides for an optimum reaction. FIG. 3 is a schematic showing a first variant of a reservoir 110 with two compartments 112 and 113, and FIG. 4 shows a second variant a reservoir 210 with three compartments 212, 213, and 216. Depending on the operation of the electrical energy production device 2, but also on the type of contents in the reservoir 10, 110, or 210 (state of the various compounds, etc.), the various compartments may include a solvent (water, for instance), electrolytes, enzymes, mediators, cofactors, a substrate (for example, glucose), or enzyme orientation molecules. The retention means 14 may be or may comprise a seal 16.

It is also possible for the reservoir to include two distinct spaces, each comprising a plurality of compartments.

Other Elements Constituting the Electrical Energy Production Device

The electrical energy production device 2 may also include the usual elements of electrochemical cells and particularly fuel cells. Thus, the device may comprise conductive elements in contact with an anode (in particular on the side opposite the side of the anode in contact with the diffusion layer). When the device is supplied with a gas, means for diffusing this gas may be arranged to allow the gas to be supplied.

Lastly, the electrical energy production device may comprise a substrate, preferably a quite rigid one, and a trim element, such as a strip made of fiberglass, plastic, or polystyrene, or preferably a biosourced material, surrounding the assembly of the components described above, with the exception of the reservoir 10, which is accessible so that the contents thereof may be released. The purpose of this element is to secure and protect the device.

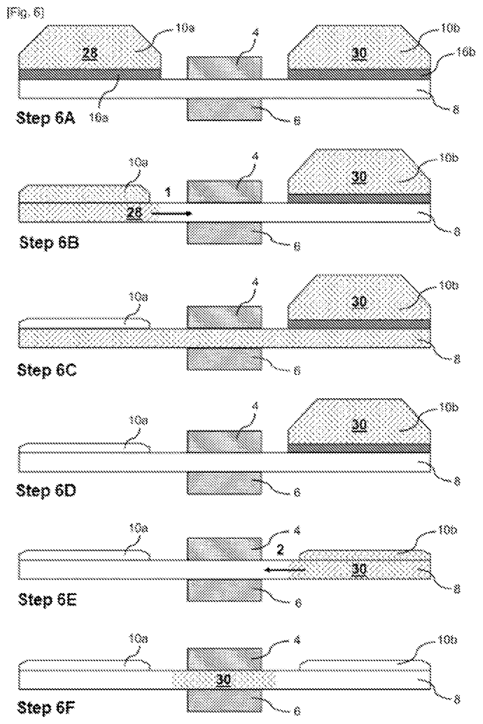

FIG. 5 shows a first variant of the device according to the invention in which two reservoirs 10a and 10b are used. Once again, these reservoirs may be of the types described above. Here, the reservoir 10a comprises a breakable seal 16 and liquid 28. The reservoir 10b is located on the other side of the cell and comprises piercing means located outside the chamber of the reservoir 10b for piercing the seal 16. The reservoir 10b does not include any liquid 28. The diffusion means 8 goes from a reservoir 10a to the reservoir 10b and is in contact with both.

According to this embodiment, it is possible to activate and then deactivate the electrical energy production device 2.

Steps 5A to 5C correspond to steps 1A to 1D described earlier with release of a liquid 28 into the diffusion layer 8 and electricity production according to a reactivatable operation.

It is possible to deactivate the electrical energy production device 2 as shown in steps 5D and 5E. In step 5D, a pressure is exerted on the reservoir 10b in order to open this reservoir 10b, which does not contain any liquid to be released. Opening may be achieved by the piercing means 18 coming into contact with the retention means 14. Since the reservoir 10b is then in fluid communication with the diffusion layer 8, the liquid 28 may enter the reservoir 10b. Means for forcing the liquid 28 to at least partially enter the reservoir 10b are used, as shown in step 5E. These means may be, for example, gravity, a flow of gas such as air (if the reservoir 10b contains a partial vacuum). Once a sufficient quantity of the liquid 28 has been absorbed or transferred into the reservoir 10b so that there is no longer enough liquid 28 between the anode 4 and the cathode 6, the electrical energy production device 2 is deactivated. Naturally, the quantity of liquid 28 must be predetermined to enable deactivation.

The deactivation means comprising the reservoir 10b may be temporary. It is possible to reactivate the electrical energy production device 2 so as to send the liquid 28 back to the anode 4 and the cathode 6. This can be done by gravity, for example, by repositioning the reservoir 10b or by again pressing on the ampoule 10b so as to reinject the liquid 28 toward the anode and cathode.

Alternatively, it is possible as indicated earlier for the reservoir 10b to include one or more compounds for deactivating the electrical energy production device 2 as a function of the contents of the solution 28. Various deactivation strategies may be implemented. For example, these may consist of the presence of a compound absorbing the liquid 28, a change in pH through the introduction of an acid or a base, a change of temperature, breakage of a secondary/tertiary structure by adding an organic solvent, adding an enzyme inhibitor decreasing the activity of the enzymes by fixing said enzymes, or adding salt to stop hydration of the enzymes. Other strategies are possible, especially depending on the nature of the liquid 28 and the quantity thereof in the reservoir 10a.

For instance, FIG. 6 shows a second embodiment variant of the invention showing an electrical energy production device 2 that can be used several times, i.e., a multi-use device. According to this embodiment, it is possible to activate the electrical energy production device 2 several times by arranging a plurality of reservoirs which can release, each in turn, the activating liquid 28 of the device.

Once again, these reservoirs may be of the types described above. Here, each reservoir 10a and 10b comprises a seal (16a and 16b) which can be broken simply by pressure. Other means for breaking these retention means, such as for example spikes as described earlier, are obviously possible.

According to this embodiment, steps 6A to 6C correspond to steps 1A to 1D, with release of the solution 28A into the diffusion layer 8 and electricity production according to a reactivatable operation.

In step 6D, the electrical energy production device 2 is inactive due to evaporation of the liquid or the absence of fuel. An inactivity of the electrical energy production device 2 can be defined as the total absence of energy production or when the quantity of energy produced drops below a predetermined minimum value.

In order to reactivate the electrical energy production device 2 (steps 6E and 6F), action is taken on the reservoir 10b, filled with activation liquid 28B which can subsequently be released by action (for example, the application of pressure) on the reservoir 10b. The operating principle is the same as that described previously.

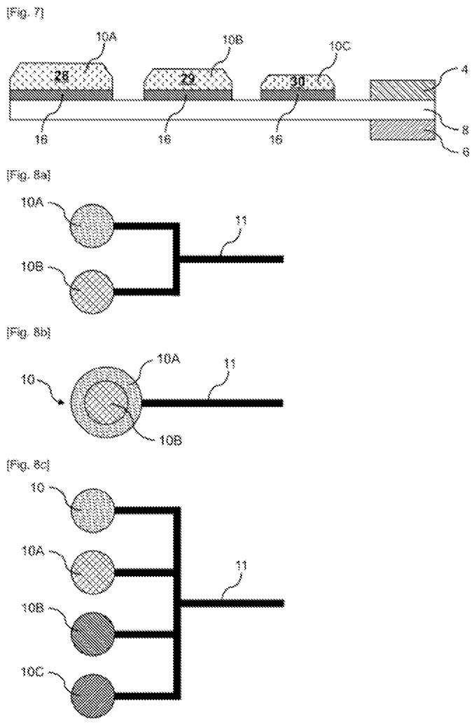

FIG. 7 shows a third embodiment of an electrical energy production device 2 that can be reactivated and/or deactivated more than once.

In this case, the electrical energy production device 2 can be activated by exerting pressure on the reservoir 10A, which breaks a seal 16 (as described earlier).

When the electrical energy production device 2 is no longer active (no activity or not enough), it is possible to reactivate the device by again injecting the activating liquid between the anode 4 and the cathode 6, by regenerating the solution already present, or, on the contrary, by injecting a deactivator. This can be done by means of the reservoirs 10B and 100, the contents of which, 29 and 30, respectively, and the size of which are determined according to the nature of the activating liquid 28 in the ampoule 10A, as well as the desired goal, increasing or maintaining activity, or decreasing or stopping energy production by the electrical energy production device 2. The contents 29 and/or 30 can therefore contain a recharge of liquid 28 or other compounds of biomolecules, electrolytes, mediators, enzymes, or substrate. It may also contain a means for stopping or decreasing electrical activity, such as a partial vacuum, an absorption agent, etc.

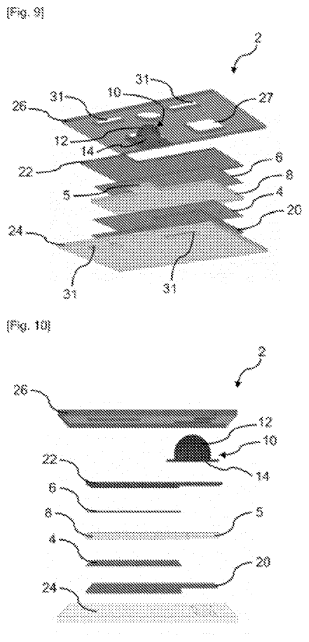

FIGS. 8a to 8c are partial schematics of the arrangement of the reservoirs 10, 10A, 10B, and/or 10C which may be included in electrical energy production devices 2 according to the invention, as described in the present application.

The partially illustrated variant shown in FIG. 8a includes two reservoirs 10 and 10A, each of which may include an identical or different composition (preferably a liquid). The two reservoirs are connected in parallel by a duct 11, which is in turn connected to or comprises or consists of a diffusion layer. The two compositions may be released by the piercing of separation means (not shown) simultaneously or at different times. In this arrangement, the reservoirs 10 and 10A are separate and distinct. The compositions that they contain can only interact with each other outside their respective reservoirs.

The variant shown in FIG. 8b comprises a reservoir 10 comprising two compartments 10A and 10B, each containing an identical or different composition. The compartment 10B can be contained entirely in the compartment 10B. Piercing means (not shown) are arranged to enable simultaneous or sequential piercing of the compartments 10A and 10B. In particular, they may enable mixing between the two compositions of the compartments 10A and 10B in the reservoir 10 before release into the duct 11.

The variant shown in FIG. 8c comprises four reservoirs 10, 10A, 10B, and 10C. Its operating principle is similar to the variant in FIG. 8a. The diagram provides an understanding of how it is possible to influence energy production by varying the nature of the compositions contained in these reservoirs: either by increasing, maintaining, or decreasing it. As in the reservoirs shown in FIG. 8a, the compositions can only interact outside their respective ampoules in the duct 11.

Example of Invention Implementation

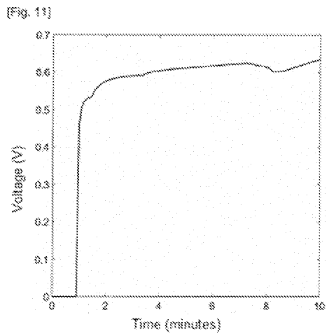

An example of the electrical energy production device 2 has been made. The device is a fuel cell. More specifically, it is a glucose biofuel cell having the structure shown in FIGS. 9 and 10. The electrodes comprise MWNT sheets (see above). These sheets have been modified by deposition (pipette) of a solution of the mediator (phenanthroline quinone, 10 mmol/L in acetonitrile) in a quantity of 80 μL/0.785 cm² at the anode 4, and of the promoter (protoporphyrin IX, 10 mmol/L in water) with a quantity of 80 μL/0.785 cm² at the cathode 6. After the two electrodes have been dried, the enzymes are added to these sheets by deposition (pipette) of a solution of 5 mg/L FAD GDH with a quantity of 80 μL/0.785 cm² at the anode 4, and a solution of 5 mg/L Bilirubin oxidase with a quantity of 80 μL/0.785 cm² at the cathode 6. Each sheet/electrode 4 and 6 was then allowed to dry for one full night at ambient temperature.

As an illustration, a reservoir 10 made by reusing a medication packaging (plastic blister pack closed with an aluminum foil) was filled with approximately 250 μL of a glucose solution at a concentration of 150 mM in a phosphate buffered saline (PBS) solution at a concentration of 0.1 M. It was then covered with a polyethylene film (brand name PARAFILM M), which is a plastic paraffin film on paper made by the company Bemis North America located in Neenah, Wisconsin (United States). This is a thermoplastic material (which consequently cannot be used in an autoclave) that is ductile, malleable, waterproof, odorless, cohesive, and translucent. The reservoir 10 is then sealed with an adhesive strip to prevent any undesirable leaking of the glucose solution.

After the electrodes 4 and 6 have been dried, the electrical energy production device shown in FIGS. 1 and 2 is assembled as follows: A sheet of blotting paper such as Whatman filter paper with dimensions depending on the configuration of the biofuel cell, a thickness of 190 μm, and a weight of 97 g/m⁻² constituting the diffusion layer 8, is sandwiched between the two electrodes 4 and 6. This layer of blotting paper comprises an extension 5. A GDL (Gas Diffusion Layer) graphite sheet constituting a conductive layer 20 is placed on the side of the anode 4 that is not the side in contact with the diffusion layer 8.

A conductive and gas diffusion layer 22 also consisting of a sheet of graphite is brought into contact with the cathode 6 (on the side opposite the side of the cathode 6 in contact with the diffusion layer 8). This layer allows oxygen to be brought to the cathode 6. It also constitutes a conductive layer. Diffusion of the gas is achieved by means of a recessed line allowing the gas to flow. As can be seen in FIG. 9, the gas diffusion layer 22 and the cathode 6 are sized and arranged so as to place the retention means 14 of the reservoir 10 and the diffusion layer 8 directly in front of each other to allow the contents of the reservoir 10 to diffuse into the diffusion layer 8 when the retention means 14 is pierced.

Lastly, the electrical energy production device 2 comprises a substrate 24, preferably one that is quite rigid, made of polyester or paper, for example, and a trim layer 26 consisting of a fiberglass strip (or another material, preferably biosourced) surrounding all the components described above, with the exception of:
the ampoule 10 accessible for releasing the contents thereof,
the openings 27 placed in front of the gas diffusion layer 22 and allowing oxygen to reach the cell; and
possible openings 31 giving access to the electrical conductive layers 20 or 22.

When electricity production is required, pressure is exerted on the shell 12 of the reservoir 10, enough pressure to break the retention means 14 and release the glucose solution onto the extension 5 of the sheet of blotting paper or the diffusion layer 8. The liquid spreads into the sheet by capillary action and allows for an ion exchange of protons between the cathode and the anode and consequently the production of current.

FIG. 11 shows the measurement of energy produced by the electrical energy production device 2 described above. The measurement is taken using a potentiostat with the ends of the counter-electrode and the reference electrode being short-circuited together and connected to the anode, while the working electrode is connected to the cathode by means of connectors such as alligator clamps, not shown. The open circuit potential (OCP) is then measured.

As an illustration, the retention means 14 of the reservoir 10 is broken at t=50 seconds by manual compression of the shell 12 thereof, which allows the contents thereof to spread into the diffusion layer 8. Ten seconds later (t=60 seconds) and 25 seconds later (t=75 seconds), the potentiostat registers a voltage of 0.458 V and 0.526 V, respectively. As shown in FIG. 11, the electrical energy production device 2 continues to produce electricity over time.

The invention is not limited to the presented embodiments and other embodiments will be clear to a person skilled in the art.

In particular, it is possible to use materials other than those mentioned above to make the various components of the electrical energy production device.

The compounds for producing energy may also be different from those mentioned above.

### LIST OF NUMERICAL REFERENCE SIGNS AND DOCUMENT REFERENCES

2: electrical energy production device
4: anode
5: extension of the diffusion layer 8
6: cathode
8: diffusion layer or separator
10, 110, 210, 10a, 10b, 10c, 10A, 10B, and 100: reservoir
12: reservoir shell
14: retention means
16: seal
18: piercing means
20: conductive layer
22: gas diffusion and conductive layer
24: substrate
26: trim layer
27: opening allowing oxygen to enter
28, 29, 30: liquid contained in the reservoir
31: openings for access to the conductive sheets 20 and 22
112, 113, 212, 213, 216: reservoir compartments
(I) Lockhart, H.; Paine, F. A. Packaging of Pharmaceuticals and Healthcare Products; Springer US: Boston, Mass., 1996.
(2) Roggenhofer, A. The Magic Triangle of Blister Packaging. Pharmaceutical Processing. 2013, pp 34-36.
(3) Raina, H.; Jindal, A. Packaging of Non-Injectable Liquid Pharmaceuticals: A Review. J. Appl. Pharm. Sci. 2017, 7 (2), 248-257.
(4) Forcinio, H. Blister Packaging Moves Forward. Pharmaceutical Technology Europe. 2017, pp 16-19.
(5) Kelso, D. M.; Agarwal, A.; Sur, K. US20120107811A1: Burstable Liquid Packaging and Uses Thereof., 2012.
(6) Brettschneider, T.; Czurratis, D.; Grimm, A. GB2538846A: Storage Unit, Method for Manufacturing a Storage Unit and Method for Releasing Fluid Stored in a Storage Unit, 2016.
(7) Kurowski, D.; Paul, O. WO2012062648A1: Blister Packaging for Liquid and Use Thereof and Method for Supplying Liquid to a Fluidic Assembly, 2012.
(8) Kurowski, D.; Paul, O. US20130327672A1: Blister Packaging for Liquid and Use Thereof and Method for Supplying a Liquid to a Fluidic Assembly, 2013.
(9) Stange, O.; Höhl, H.-W.; Diederich, R.; Droder, K.; Herrmann, C.; Dietrich, F.; Blumenthal, P.; Stühm, K.; Bobka, P.; Schmidt, C.; et al. WO2017032674: Blister Packaging, 2015.
(10) Wright, D. W.; Aiello, D.; Kroehl, P.; Kayyem, J. F.; Gray, D. S. U.S. Pat. No. 9,598,722B2: Cartridge for Performing Assays in a Closed Sample Preparation and Reaction System., 2017.

1. A device for producing and/or storing electrical energy, the device comprising:
an anode,
a cathode,
a separator allowing for the transfer of at least one compound capable of triggering and/or enabling a production and/or storage of electrical energy, arranged between the anode and the cathode, and
at least one breakable, pierceable, and/or deformable reservoir made of a compound capable of triggering and/or enabling a production and/or a storage of electrical energy, said reservoir having means for bringing said compound and said separator into contact with each other; said means for bringing said compound and said separator into contact with each other being means for transferring a liquid.

2. The device according to claim 1 wherein the anode and/or the cathode comprises an enzyme.

3. The device according to claim 1 wherein the reservoir comprises a shell having an opening and retention means blocking the opening of the shell.

4. The device according to claim 1 wherein the means for bringing the compound capable of triggering a production and/or storage of electrical energy and the separator into contact with each other include piercing or opening means.

5. The device according to claim 1 wherein said reservoir of the device comprises one or more compartments.

6. The device according to claim 1, further comprising at least one other reservoir, said at least one other reservoir comprising said compound capable of triggering a production and/or a storage of electrical energy or another compound.

7. The device according to claim 1 wherein said compound capable of triggering a production and/or a storage of electrical energy is a liquid, a solid, or a gel.

8. The device according to claim 1 wherein said means for bringing said compound and the separator into contact with each other comprises a duct and/or an extension of said separator.

9. The device according to claim 1, further comprising means for increasing, decreasing, deactivating, and/or reactivating the production and/or storage of electrical energy.

10. The device according to claim 1, further comprising one or more activation means.

11. (canceled)

12. A disposable device comprising the electrical energy production and/or storage device according to claim 1.

13. The disposable device according to claim 12 wherein said disposable device is a medical test.

14. A kit for manufacturing an energy production and/or storage device, said kit comprising the device according to claim 1 and usage instructions.

15. The device according to claim 4 wherein said piercing or opening means comprises at least one component having a cutting or pointed end.

16. The device according to claim 7 wherein said compound capable of triggering a production and/or a storage of electrical energy is an aqueous liquid.

17. The device according to claim 8 wherein said extension or said duct is configured to come into contact with a part of the reservoir.

18. The device according to claim 10 wherein said one or more activation means comprises a switch.

19. The device according to claim 18 wherein said switch comprises a removable tab.

20. The disposable device according to claim 13 wherein said medical test is a pregnancy test.
